(19) United States
(12) Patent Application Publication
(10) Pub. No.: US 2013/0160520 A1
(43) Pub. Date: Jun. 27, 2013

(54) GAS ANALYZER

(71) Applicants: Tomohisa Kawata, Osaka-shi (JP); Mieko Otonashi, Osaka-shi (JP); Mikihiro Yamanaka, Osaka-shi (JP); Keita Hara, Osaka-shi (JP)

(72) Inventors: Tomohisa Kawata, Osaka-shi (JP); Mieko Otonashi, Osaka-shi (JP); Mikihiro Yamanaka, Osaka-shi (JP); Keita Hara, Osaka-shi (JP)

(21) Appl. No.: 13/522,232

(22) PCT Filed: Dec. 1, 2010
(86) PCT No.: PCT/JP2010/071466

§ 371 Date: Jul. 13, 2012

(30) Foreign Application Priority Data
Feb. 5, 2010 (JP) 2010-024459

(51) Int. Cl. G01N 33/497

(52) U.S. Cl. CPC G01N 33/497
USPC 73/23.3

(57) ABSTRACT

A gas analyzer having a compact and simple structure and having a high performance in separating components of a sample gas is provided. The gas analyzer according to the present invention includes a gas introduction unit including a gas introduction port for introducing a sample gas; a gas separation unit including a microcolumn for separating components of the sample gas supplied from the gas introduction unit; and a gas detection unit detecting a gas component separated by the gas separation unit. The microcolumn is provided with an internal channel having a wall surface modified by a stationary phase. This stationary phase is made of a polar material having a relative permittivity of not less than 10 at 30° C.

### TECHNICAL FIELD

The present invention relates to a gas analyzer, and particularly to a gas analyzer detecting a small amount of gas component contained in a sample gas with high accuracy.

### BACKGROUND ART

In our country, population decrease and aging combined with declining birthrate are progressing, which leads to a rapid increase in the proportion of the elderly people aged 65 and over to the total population. Specifically, it is estimated that approximately one in four corresponding to about 25.2% of the total population will be an elderly person in 2013, and approximately one in three corresponding to about 33.7% of the total population will be an elderly person in 2035. Since elderly people are more likely to depend on medical institutions, a burden on medical care is expected to increase in the future.

For younger people, there have been a significant improvement in a living environment, decreased opportunities for physical exercise due to development in IT technology and the like, which leads to a problem such as metabolic syndrome. Consequently, the population of the younger people suffering from lifestyle-related diseases and the like is increasing each year. Thus, younger people are also increasingly utilizing medical service in recent years.

In consideration of the above-described trend, it is also said that a burden on medical care will reach its limitation in a few years. Thus, it is desired to minimize a burden of medical care. In recent years, an attention has been paid particularly to preventive healthcare that may prevent dependence on medical institutions.

By fully developing the preventive healthcare, it becomes possible to prevent people from suffering from diseases, so that the number of people suffering from diseases can be decreased. If the number of people suffering from diseases is decreased by using such an approach, it is advantageous not only in that the burden on medical care can be lessen, but also in that the medical expenses can be reduced with current concerns about the collapse of the medical insurance system.

Thus, in order to fully develop preventive healthcare, it is desirable to achieve widespread use, in every household, of a system for individuals to obtain their own health information for easily managing their health using devices at hand. Examples of indicators for obtaining health information include biological samples such as blood pressure, blood, urine, sweat, saliva, and exhaled breath. Such a biological sample includes a plurality of substances each having a numerical value that varies depending on diseases or signs thereof like a blood sugar level in blood.

The contents of the substances contained in such a biological sample are separately measured, thereby obtaining health information. Thus, it becomes possible to accurately grasp the individuals' own health conditions. By objectively grasping the individuals' own health conditions in this way, diseases can be found at an early stage, and therefore, their lifestyles can be improved beforehand so as not to suffer from such diseases.

Among the above-mentioned biological samples, particularly, exhaled breath contains a plurality of substances each having a numerical value that varies depending on diseases or signs thereof, can be quickly and easily sampled and measured, and is also measured as gas that can be measured in a non-invasive manner, which causes less physical damage, and the like. Accordingly, daily measurement of exhaled breath may be less uncomfortable. Therefore, exhaled breath can be recognized as one of biological samples that is most suitable for daily health management.

In terms of the above-described advantages, studies for identifying diseases based on the components contained in exhaled breath have been actively conducted. With regard to the correlation between exhaled breath and diseases that has been found by past studies, it becomes apparent that the components in the exhaled breath of the patient suffering from a lung cancer are partially different from those in the exhaled breath of a healthy person.

More specifically, it is known that a person exhaling breath containing a large quantity of nitric oxide and carbon monoxide is more likely to suffer from a lung disease. From the exhaled breath of the patient suffering from asthma and a chronic obstructive pulmonary disease (COPD), nitric oxide and carbon monoxide each are detected at a high concentration.

The following is another example showing a correlation between the components in exhaled breath and diseases. For example, in the case of the exhaled breath of the patient suffering from a gastrointestinal disease such as indigestion and a duodenal ulcer, hydrogen tends to be detected at a high concentration. The exhaled breath of the patient suffering from lipid oxidation, asthma, bronchitis or the like is highly correlated to oxidant stress, in which case ethane, pentane and the like each tend to be detected at a high concentration. In this way, by measuring the concentration of each component contained in the exhaled breath, disease information can be obtained and healthcare guidance can be provided.

Among the above-described components, particularly, acetone in the exhaled breath is produced when fat (fatty acid) and protein (amino acid) are decomposed. Accordingly, acetone has been conventionally recognized as an indicator showing the degree of activity of sugar metabolism. It is known that a person who is extremely hungry as in the fasting state where no food is taken or suffers from serious diabetes exhales breath containing a relatively large quantity of acetone. It may also be considered that the decreasing amount of body fat can be clarified by grasping the amount of acetone contained in exhaled breath.

The following is an explanation of the detailed mechanism that body fat is changed into acetone which is then discharged out of the body. Specifically, ketone bodies such as acetoacetic acid, hydroxybutyric acid and acetone are first produced in blood in the fat metabolism process. Then, acetoacetic acid and hydroxybutyric acid in the produced ketone bodies are reused in organs other than a liver while acetone is discharged out of the body through a lung as exhaled breath. In addition, fat metabolism occurs by utilizing the body fat accumulated in the body as energy when glucose in the blood becomes insufficient due to consumption by food restriction or physical exercise.

In this way, acetone is produced in the body fat burning process and discharged together with the exhaled breath contained therein. Accordingly, by measuring the concentration of acetone in the exhaled breath, the burning state of body fat can be directly grasped.

In addition, as a method of separately measuring the concentrations of the plurality of components in the exhaled breath, there is a conventionally known detection method of separating the components by utilizing gas chromatography and then performing detection by a detector such as a thermal conductivity type detector, a hydrogen flame ionization type detector, an electron capture type detector, a mass spectrometric detector, or the like. The above-described detection method has an advantage that each component can be detected on the ppb-ppt level with high sensitivity.

However, conventional devices for measuring exhaled breath, that is, gas chromatography, are large in size and weight and also expensive, and further, require the user to learn how to operate the devices. Accordingly, these devices are not recognized as practical for widespread use in every household like a device provided at hand.

Furthermore, in order to accurately analyze the components contained in exhaled breath, it is necessary to remove large quantity of water vapor contained in the exhaled breath. However, since the conventional device for measuring exhaled breath is not provided with a unit performing pre-processing for removing moisture in the exhaled breath, a part of a small amount of components contained in exhaled breath is difficult to be detected, which prevents accurate analysis.

### CITATION LIST

### Patent Literature

PTL 1: Japanese Patent Laying-Open No. 2002-181674
PTL 2: Japanese Patent National Publication No. 2005-512067
PTL 3: Japanese Patent Laying-Open No. 2006-145254

### SUMMARY OF INVENTION

### Technical Problem

As an attempt to solve the above-described problems, for example, Japanese Patent Laying-Open No. 2002-181674 (hereinafter also referred to as “PTL 1”) and Japanese Patent National Publication No. 2005-512067 (hereinafter also referred to as “PTL 2”) each disclose a device for performing preprocessing of a sample to remove moisture. However, since the device disclosed in each of PTL 1 and PTL 2 needs to be provided with a moisture removing unit in addition to an analyzer, the structure of the device becomes complicated, with the result that the entire device may be increased in size.

Furthermore, Japanese Patent Laying-Open No. 2006-145254 (hereinafter also referred to as “PTL 3”) discloses a technique of detecting water by using a column of normal gas chromatography. However, since the method disclosed in PTL 3 requires use of a gas chromatography device, it is almost impossible to employ this method in each household and the like.

The present invention has been made in light of the above-described circumstances. The present invention aims to provide a gas analyzer having a compact and simple structure and also having high performance in separating components of a sample gas.

### Solution to Problem

A gas analyzer according to the present invention includes a gas introduction unit into which a sample gas is introduced through a gas introduction port; a gas separation unit including a microcolumn for separating components of the sample gas supplied from the gas introduction unit; and a gas detection unit detecting a gas component separated by the gas separation unit. The microcolumn is provided with an internal channel having a wall surface modified by a stationary phase. The stationary phase is made of a polar material having a relative permittivity of not less than 10 at 30° C.

It is preferable that the polar material is made of polyethylene glycol having an average molecular weight of not less than 200 and not more than 1000. An inner diameter of the internal channel is defined as D and a thickness of the stationary phase is defined as t, which leads to a condition that 0.005≦t/D≦0.02. It is preferable that the stationary phase has a thickness of not less than 1 μm and not more than 2 μm. It is preferable that the sample gas contains acetone.

It is preferable that the gas detection unit is provided therein with a gas sensor for detecting a detection gas and the gas sensor is disposed near an outlet port for the gas component separated by the gas separation unit.

### Advantageous Effects of Invention

The gas analyzer according to the present invention has the above-described configuration, thereby implementing a compact and simple structure, and also achieving an effect that components of the sample gas can be accurately separated to allow highly accurate detection of a small amount of gas component contained in the sample gas.

### BRIEF DESCRIPTION OF DRAWINGS

FIG. 1(a) is a schematic diagram showing an example state of a gas analyzer according to the present invention, and FIG. 1(b) is a schematic diagram showing another state of the gas analyzer according to the present invention.

FIG. 2(a) is a schematic diagram showing an example state of the gas analyzer according to the present invention, and FIG. 2(b) is a schematic diagram showing another state of the gas analyzer according to the present invention.

FIG. 3 is a schematic cross sectional view showing an example of a gas detection unit used in the gas analyzer according to the present invention.

FIG. 4(a) is an image of the cross section of an internal channel before being modified by a stationary phase that is taken by a digital microscope, and FIG. 4(b) is an image of the cross section of the internal channel after being modified by the stationary phase that is taken by the digital microscope.

FIG. 5 is a chromatogram obtained when a mixture gas of acetone/ethanol/water is introduced into a microcolumn in Example A1.

FIG. 6 is a chromatogram obtained when a mixture gas of acetone/ethanol/water is introduced into a microcolumn in Example A2.

FIG. 7 is a chromatogram obtained when a mixture gas of acetone/ethanol/water is introduced into a microcolumn in Example A3.

FIG. 8 is a chromatogram obtained when a mixture gas of acetone/ethanol/water is introduced into a microcolumn in Example A4.

FIG. 9 is a chromatogram obtained when a mixture gas of acetone/ethanol/water is introduced into a microcolumn in Example A5.

FIG. 10 is a chromatogram obtained when a mixture gas of acetone/ethanol/water is introduced into a microcolumn in Comparative Example A1.

FIG. 11 is a graph showing an output of the resistance change detected by a gas sensor.

FIG. 12 is a graph showing an output of the resistance change at the time when a sample gas is introduced into a gas analyzer in Example 3 at a pressure of 0.04 MPa.

FIG. 13 is a graph showing an output of the resistance change at the time when the sample gas is introduced into the gas analyzer in Example 3 at a pressure of 0.11 MPa.

FIG. 14 is a graph showing an output of the resistance change at the time when the sample gas is introduced into the gas analyzer in Example 3 at a pressure of 0.26 MPa.

FIG. 15 is a graph showing an output of the resistance change at the time when the sample gas containing 1 ppm of acetone is introduced into a gas analyzer in Example 4.

FIG. 16 is a graph showing an output of the resistance change at the time when the sample gas containing 1 ppm of ethanol is introduced into the gas analyzer in Example 4.

FIG. 17 is a graph showing an output of the resistance change at the time when the sample gas containing 1 ppm of ethanol and 1 ppm of acetone is introduced into the gas analyzer in Example 4.

FIG. 18 is a graph showing an output of the resistance change at the time when exhaled breath is introduced as a sample gas into the gas analyzer in Example 4.

FIG. 19 is a graph showing a change over time of component separation at the time when the mixture gas is separated using the microcolumn in Example A1.

FIG. 20 is a graph showing a change over time of component separation at the time when the mixture gas is separated using the microcolumn in Example A2.

### DESCRIPTION OF EMBODIMENTS

The embodiments of the present invention will be hereinafter described with reference to the accompanying drawings. The configuration shown in the figures and set forth in the following description is merely by way of example and the scope of the present invention is not limited to that shown in the figures and set forth in the following description. In the accompanying drawings of the present invention, the same or corresponding components are designated by the same reference characters.

Furthermore, in the accompanying drawings of the present application, the dimensional relationship such as length, width and thickness is modified as appropriate for the purpose of clarifying and simplifying each figure, and is not to actual scale.

<<Gas Analyzer>>

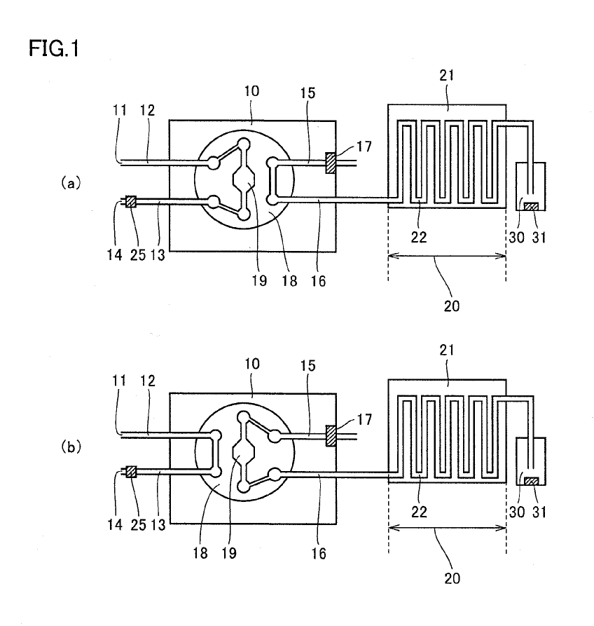

FIG. 1(a) is a schematic diagram showing an example state of a gas analyzer according to the present invention, and FIG. 1(b) is a schematic diagram showing another state of the gas analyzer in FIG. 1(a). As shown in FIG. 1(a), the gas analyzer according to the present invention includes a gas introduction unit 10 including a gas introduction port 11 for introducing a sample gas; a gas separation unit 20 including a microcolumn 21 for separating components of the sample gas supplied from gas introduction unit 10; and a gas detection unit 30 detecting a gas component separated by gas separation unit 20. Microcolumn 21 is provided with an internal channel 22 having a wall surface modified by a stationary phase. This stationary phase is made of a polar material having a relative permittivity of not less than 10 at 30° C.

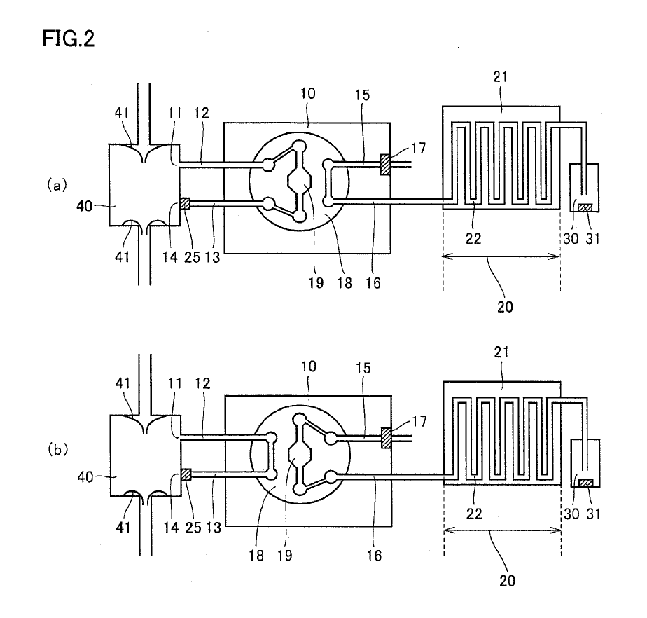

By providing the stationary phase made of such a polar material on the wall surface of internal channel 22 in microcolumn 21, components of the sample gas can be separated, thereby allowing an improvement in detection accuracy of the gas analyzer. In the following description, an example of the operation of the gas analyzer according to the present invention will be explained with reference to FIGS. 2(a) and 2(b).

FIG. 2(a) is a schematic diagram showing an example state of the gas analyzer according to the present invention, and FIG. 2(b) is a schematic diagram showing another state of the gas analyzer according to the present invention. In the gas analyzer according to the present invention, in the state shown in FIG. 2(a) (which will be hereinafter also referred to as the “first state”), the sample gas is introduced through an introduction port of a gas sampling unit 40, and supplied from gas introduction port 11 to a gas storage unit 19 through a first channel 12. Then, the sample gas is discharged from gas storage unit 19 through a second channel 13 and a gas discharge port 14 to gas sampling unit 40. The flow velocity of such a sample gas is controlled by airflow generating means 25.

On the other hand, in the first state, a third channel 15 supplying a carrier gas is directly connected to a fourth channel 16 connected to microcolumn 21 in gas separation unit 20. Then, the carrier gas having a flow velocity adjusted by pressure adjusting means 17 flows from third channel 15 through fourth channel 16 and then through microcolumn 21 in gas separation unit 20.

Then, channel switching mechanism 18 is used to change the connection relationship between first channel 12 and second channel 13 and between third channel 15 and fourth channel 16 to switch the first state to the second state as shown in FIG. 2(b). In this second state, as shown in FIG. 2(b), third channel 15, gas storage unit 19 and fourth channel 16 are connected. By switching the first state to the second state in this way, the sample gas in gas storage unit 19 introduced from first channel 12 is supplied to microcolumn 21 in gas separation unit 20 through fourth channel 16 together with the carrier gas supplied from third channel 15.

The sample gas supplied to gas separation unit 20 is repeatedly adsorbed onto and desorbed from the stationary phase on the wall surface of internal channel 22 in microcolumn 21, in which ease of adsorption and desorption varies depending on the components of the sample gas. Accordingly, the components of the sample gas can be separated in such a manner that the more the components are absorbed onto the stationary phase, the slower the moving velocity is while the less the components are absorbed onto the stationary phase, the faster the moving velocity is.

When the sample gas passes through gas separation unit 20, the separated gas components of the sample gas are sequentially introduced into gas detection unit 30. Each of these components is sensed by a gas sensor 31 of gas detection unit 30. The time period from the time when the sample gas is introduced into the gas analyzer until the time when gas sensor 31 senses a gas component is hereinafter referred to as a retention time. This retention time shows a value inherent in the component of the sample gas. Based on this retention time, the gas component is identified. The gas analyzer according to the present invention detects gas components of the sample gas in this way. Each unit constituting the gas analyzer of the present invention will be hereinafter described in detail.

<Gas Introduction Unit>

In the present invention, gas introduction unit 10 is provided for supplying a part of the sample gas to gas separation unit 20. Such gas introduction unit 10 is not limited only to the structure as shown in FIG. 1, but it is preferable that, for example, a switching port is provided in the channel for supplying the sample gas into gas separation unit 20. Gas introduction unit 10 may have any structure as long as it can adjust, by operating this switching port, the flow velocity of the sample gas supplied to the gas separation unit. An example of gas introduction unit 10 will be hereinafter described with reference to FIGS. 2(a) and 2(b).

Gas introduction unit 10 shown in FIG. 2(a) includes first channel 12 for introducing the sample gas through gas introduction port 11, second channel 13 for discharging a part of the introduced sample gas from gas discharge port 14, and also gas storage unit 19 for connecting first channel 12 and second channel 13 to each other and storing the sample gas therein.

On the other hand, gas introduction unit 10 includes third channel 15 for introducing a carrier gas and fourth channel 16 for supplying a sample gas to gas separation unit 20, which are provided separately from first channel 12 and second channel 13. It is to be noted that, in the first state shown in FIG. 2(a), third channel 15 and fourth channel 16 are directly connected to each other without through gas storage unit 19. Accordingly, the carrier gas introduced into third channel 15 is supplied to gas separation unit 20 through fourth channel 16. In the first state, the sample gas is not supplied to gas separation unit 20, but the sample gas introduced from first channel 12 passes through gas storage unit 19. Then, a part of the sample gas is stored in gas storage unit 19 while the remainder thereof is discharged from gas discharge port 14 through second channel 13.

(Channel Switching Mechanism)

Channel switching mechanism 18 is provided in gas introduction unit 10 in order to switch the first state in which gas storage unit 19 is connected to first channel 12 and second channel 13 to the second state in which gas storage unit 19 is connected to third channel 15 and fourth channel 16. Channel switching mechanism 18 switches the first state shown in FIG. 2(a) to the second state shown in FIG. 2(b). In the second state, the sample gas stored in gas storage unit 19 in the above-described first state flows through fourth channel 16 together with the carrier gas supplied through third channel 15, and then, is supplied through this fourth channel 16 to gas separation unit 20.

Then, in the second state, when no sample gas is stored in gas storage unit 19, or when the sample gas is fully supplied to gas separation unit 20, channel switching mechanism 18 switches the second state to the first state. When the second state is switched to the first state, the sample gas is again introduced into gas storage unit 19 through first channel 12. By alternately switching between the first state and the second state in this way, the sample gas of a suitable flow rate can be introduced into gas separation unit 20 with proper timing.

In addition, in the second state shown in FIG. 2(b), first channel 12 and second channel 13 are directly connected to each other without through gas storage unit 19. Accordingly, the sample gas introduced into first channel 12 in the second state is discharged to gas sampling unit 40 through second channel 13.

(Pressure Adjusting Means)

It is preferable that third channel 15 is provided with pressure adjusting means 17, which allows control of the flow velocity of the carrier gas flowing through third channel 15. By controlling the flow velocity of the carrier gas in this way, the sample gas of constant flow rate can be supplied to gas separation unit 20 together with the carrier gas.

The flow velocity of the sample gas controlled by such pressure adjusting means 17 is not particularly limited and may be any velocity, but preferably, not less than 10 cm/sec and not more than 100 cm/sec. It is to be noted that this preferable flow velocity is different depending on the length and the cross-sectional area of the internal channel. For example, when the internal channel has a length of 10 m and a cross-sectional area of 0.04 mm², the flow velocity is, among the above-described value range, more preferably not less than 10 cm/sec and not more than 50 cm/sec, and further preferably not less than 10 cm/sec and not more than 30 cm/sec. When the internal channel has a length of 17 m and a cross-sectional area of 0.04 mm², the flow velocity is more preferably not less than 40 cm/sec and not more than 90 cm/sec, and further preferably not less than 50 em/sec and not more than 70 cm/sec. When the flow velocity of the sample gas is less than 10 cm/sec, the time required for gas detection is lengthened, which is not preferable in light of the specifications of the analyzer. The flow velocity of the sample gas exceeding 100 cm/sec is too fast, with the result that the components of the sample gas cannot be easily separated by the subsequent gas separation unit 20.

Such pressure adjusting means 17 may be any means as long as it can adjust the gas pressure, and can be, for example, a compressor, a valve, a pump, a regulator, a gas cylinder, and the like. When a compressor, a pump or the like is used, the sample gas can be supplied to gas separation unit 20 in the state where the pressurized air is adjusted with a pressure reducing valve. It is to be noted that examples of carrier gas may include an inert gas such as helium, air, or the like.

(Gas Sampling Unit)

In the gas analyzer according to the present invention, it is preferable that gas sampling unit 40 is connected to gas introduction port 11 and gas discharge port 14, as shown in FIG. 2(a). Gas sampling unit 40 is connected in this way, so that the sample gas can be efficiently introduced into gas introduction port 11 while the space storing the sample gas can be provided.

Furthermore, such space in the gas sampling unit also serves as a circulation channel through which the sample gas circulates via gas sampling unit 40, first channel 12, gas storage unit 19, and second channel 13.

It is preferable that the introduction port of gas sampling unit 40 has a component such as a mouthpiece and a mask with which the user's mouth is brought into contact so as to allow the sample gas to be directly introduced into this introduction port. By providing a mouthpiece, a mask or the like in this way, the sample gas can be readily introduced into gas sampling unit 40.

Then, it is preferable that gas sampling unit 40 is provided with a check valve 41 at each of the inlet port and the outlet port of the sample gas. When gas sampling unit 40 is provided with check valve 41 in this way, a part of the sample gas is discharged from the gas sampling unit while the remainder thereof can be circulated through gas sampling unit 40, first channel 12, gas storage unit 19, and second channel 13.

Although FIGS. 2(a) and 2(b) each show the case where the sample gas is introduced into gas introduction unit 10 using gas sampling unit 40, the method of introducing the sample gas into gas introduction unit 10 is not limited to the that using gas sampling unit 40, but a bag may be directly connected to gas introduction port 11 to introduce the sample gas into gas introduction unit 10.

(Airflow Generating Means)

It is preferable that airflow generating means 25 is provided in one or both of gas introduction port 11 and gas discharge port 14. Airflow generating means 25 is provided in this way, thereby allowing the sample gas to circulate through gas sampling unit 40, first channel 12, gas storage unit 19, and second channel 13 and also allowing the flow velocity of the sample gas flowing therethrough to be controlled. In addition, the flow velocity of the sample gas controlled by airflow generating means 25 is particularly not limited and may be any velocity, which is preferably not less than 1 mL/min and not more than 10 mL/min.

<Gas Separation Unit>

According to the present invention, gas separation unit 20 is provided for separating various gas components contained in the sample gas introduced from gas introduction unit 10, and specifically, characterized by separating the components of the sample gas using microcolumn 21 provided in gas separation unit 20. By using microcolumn 21, the gas analyzer can be reduced in size and weight.

The “microcolumn” referred herein means a chromatographic column in the shape of a chip that is provided with a microscopic channel having a width and a depth in micro order. The outer shape of such a microcolumn is not particularly limited. For example, this microcolumn may be configured using a substrate such as an Si wafer to have an outer shape of several millimeters to several tens of centimeters in length and width and to have a thickness of about several millimeters to several centimeters.

In addition, “separating the components” of the sample gas means not only the case where all of the components constituting the sample gas arc separated for each component, but also the case where any one of the components constituting the sample gas is separated from at least one of other components. In other words, when the sample gas contains three or more components, the effect of separating the components of the sample gas can be achieved as long as at least one component of three or more components is separated from two or more other components, which falls within the scope of the present invention.

It may also be considered that gas separation unit 20 includes a packed column filled with carriers each coated with a stationary phase, a capillary column having an inner wall coated with a stationary phase, and the like as a chromatographic column other than a microcolumn. However, since these chromatographic columns need to be provided with a large constant temperature bath in order to control the temperature, the gas analyzer itself may be increased in size, which does not comply with the desired purpose, which is therefore not preferable.

In the present invention, microcolumn 21 is provided with internal channel 22 having a wall surface modified by a stationary phase. This stationary phase is made of a polar material having a relative permittivity of not less than 10 at 30° C. Such a polar material having a relative permittivity of not less than 10 has a strong polarity, which allows a significant delay in the flow velocity of the polar substance, particularly, such as water, so that the components of the sample gas can be separated.

Examples of such polar material having a relative permittivity of not less than 10 may include ethylene glycol, propylene glycol, polypropylene glycol, and the like, for example, in addition to polyethylene glycol having an average molecular weight of not more than 1000. It is to be noted that the relative permittivity of the material forming a stationary phase is set at a value that is calculated using a permittivity measuring device.

It is considered that the stronger polarity the stationary phase has, the greater difference is caused in the flow velocity of the sample gas flowing through the microcolumn due to the polarity difference between the components in the sample gas, with the result that the components of the sample gas can be readily separated. Also, the greater polarity the stationary phase has, the higher the relative permittivity of the material is likely to rise. According to such relationship between the relative permittivity and the polarity, the material forming the stationary phase has a relative permittivity that is more preferably not less than 11, and further preferably, not less than 13. The material forming a stationary phase having a relative permittivity less than 10 is not preferable since the components of the sample gas cannot be separated.

In this case, it is more preferable to employ polyethylene glycol (which will be hereinafter referred to as “PEG”) having an average molecular weight of not less than 200 and not more than 1000 as a material of the stationary phase effective to separate components of the sample gas. PEG has a tendency that the greater the average molecular weight is, the higher the viscosity is and the lower the polarity is while the smaller the average molecular weight is, the lower the viscosity is and the stronger the polarity is. Accordingly, in terms of the balance between the viscosity and the polarity of PEG, it is further preferable to employ PEG having a relative permittivity of 13.7 at 30° C. and an average molecular weight of approximately 600 (PEG 600).

When the average molecular weight of polyethylene glycol is less than 200, its viscosity is relatively low, with the result that the stationary phase is hard to be retained on the wall surface of internal channel 22 in the microcolumn. When the average molecular weight of polyethylene glycol exceeds 1000, polarity is not sufficient, with the result that the separation performance for the sample gas tends to be deteriorated.

Assuming that internal channel 22 has a width of D and the stationary phase has a thickness of t, it is preferable that 0.005≦t/D≦0.02. By setting the width of internal channel 22 and the thickness of the stationary phase to comply with such a value range, the efficiency of separating the components of the sample gas can be improved. Furthermore, the microcolumn may include temperature control means, which allows the temperature of the microcolumn to be kept constant, so that more accurate separation of the components can be achieved.

Furthermore, for the purpose of improving the separation performance for each component in the sample gas, it is preferable that the thickness of the stationary phase is not less than 1 μm and not more than 2 μm.

The thickness of the stationary phase used herein is calculated by directly measuring the cross section of the microcolumn provided with the internal channel having the wall surface modified by a stationary phase, based on the image obtained when observing this cross section using a microscope.

The width and the depth (height) of the internal channel in the microcolumn can be set, for example, at approximately 100 to 300 μm. It is preferable that the width and the depth of the internal channel in the microcolumn is determined in consideration of the type of the target component, the flow rate of the sample gas introduced into the microcolumn, and the like.

It is also preferable that internal channel 22 has a length of not less than 3 m and not more than 20 m. Internal channel 22 having a length less than 3 m prevents sufficient separation of the components in the sample gas while internal channel 22 having a length greater than 20 m leads to an increase in time period required for measurement, both of which are not preferable.

<Production of Microcolumn>

A specific example will be given to explain the method of producing a microcolumn in the present invention. First, the photolithography technique is used to perform a microfabrication process such as blast processing to form a continuous groove on the surface of the substrate such as an Si wafer.

Then, the method such as anode bonding is used to airtightly join the substrate having a continuous groove formed thereon and a glass plate such that the surface of the substrate having the groove formed thereon faces the glass plate. Then, unmodified capillary glass is attached to one end of the formed internal channel 22, and the solution having the stationary phase dissolved therein is charged into the internal channel in the microcolumn. The solvent thereof is then removed, thereby modifying the inner wall of internal channel 22 in the microcolumn by the stationary phase.

<Sample Gas>

It is preferable that acetone is contained in the sample gas including components that are separated using the gas analyzer according to the present invention. It is difficult by the conventional gas analyzer to efficiently separate a small quantity of acetone contained in water. Even if acetone can be separated, the separation accuracy is not sufficient. On the other hand, the gas analyzer according to the present invention can entirely solve the conventional problems.

<Gas Detection Unit>

Gas detection unit 30 serves to sequentially detect the gas components separated in gas separation unit 20, for which it is preferable to use gas sensor 31. According to the present invention, gas detection unit 30 includes gas sensor 31 for detecting a chemical substance. Examples of such gas sensor 31 may include a semiconductor sensor, an electrochemical gas sensor, a QCM, an FID, and the like. Among these sensors, it is preferable to employ a semiconductor sensor since it is inexpensive and readily available.

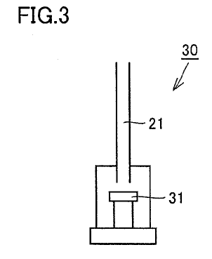

FIG. 3 is a schematic cross sectional view showing an example of the gas detection unit used in the gas analyzer according to the present invention. In the present invention, it is preferable that gas sensor 31 is disposed near the outlet port for the gas component separated by gas separation unit 20, as shown in FIG. 3. Gas sensor 31 is disposed near the outlet port for the gas component in this way, so that the sensitivity to detect the target component can be improved. The terms “near the outlet port for the gas component” used herein means that gas sensor 31 is located in the range of not less than 0.5 mm and not more than 3.0 mm from the outlet port for the gas component. In addition, “21” in FIG. 3 indicates an extension of the microcolumn as a microcolumn for convenience, and, for example, a capillary glass tube is used in practice.

Gas separation unit 20 and gas detection unit 30 are connected using a capillary glass tube, which is, however, not preferable since the capillary glass tube has a relatively small diameter, which makes it difficult for gas sensor 31 to sense the sample gas when the gas component outlet port of the capillary glass tube is located at a distance from gas sensor 31.

It is preferable that gas sensor 31 is connected to a signal receiving mechanism (not shown) such as a digital multimeter via a conducting wire and the like. Such a signal receiving mechanism needs to be configured to receive a change in the voltage value of the constant resistance of gas sensor 31 as a signal change when gas sensor 31 detects a gas component.

Furthermore, it is preferable that the signal receiving mechanism is connected to a computer. The computer referred herein means a component storing signal data detected by the signal receiving mechanism, converting the signal data into a chromatogram and providing display of the converted data. It is to be noted that the computer may be configured to have a function of the channel switching mechanism to control switching between the first state and the second state.

Although the present invention will be hereinafter described in greater detail with reference to Examples, the present invention is not limited thereto.

### Example A 1

In the present example, a microcolumn was produced by the following procedure. First, gas separation unit 20 was produced such that internal channel 22 having a width of 100 μm and a depth of 100 μm was formed in a meandering line in which neighboring lines of the channel were arranged at distance of 100 μm from each other.

Specifically, a 4-inch silicon wafer was subjected to photolithography processing and then blast processing, to thereby form a groove having a width of 100 μm and a depth of 100 μm in a meandering line in which neighboring lines of the groove were arranged at a distance of 100 μm from each other. Then, using anode bonding, a glass plate of 4 inch square was closely adhered to the surface of the silicon substrate on which the groove was formed. Then, dicing was performed to form a microcolumn of 4 cm square.

The entire length of internal channel 22 formed in this way was 9 m. The introduction port and the discharge port of internal channel 22 in this gas separation unit 20 were then attached with a capillary glass having an outer diameter of 0.35 mm and an inner diameter of 0.25 mm that had an unmodified surface.

On the other hand, a 1.0% acetone solution was prepared that was obtained by dissolving, in acetone, polyethylene glycol having an average molecular weight of 600 and a relative permittivity of 13.74 at 30° C. (PEG 600: manufactured by GL Sciences, Inc.). Such a 1.0% acetone solution was introduced through the introduction port of the microcolumn in gas separation unit 20 to fill the internal channel with the acetone solution.

Then, a hot plate was used to raise the temperature of gas separation unit 20 to 80° C., which was held for ten minutes, thereby evaporating most acetone within internal channel 22. After evaporating most acetone in this way, a diaphragm-type dry vacuum pump DA-15D (manufactured by ULVAC KIKO Inc.) having a solvent trap was connected to the introduction port side of internal channel 22.

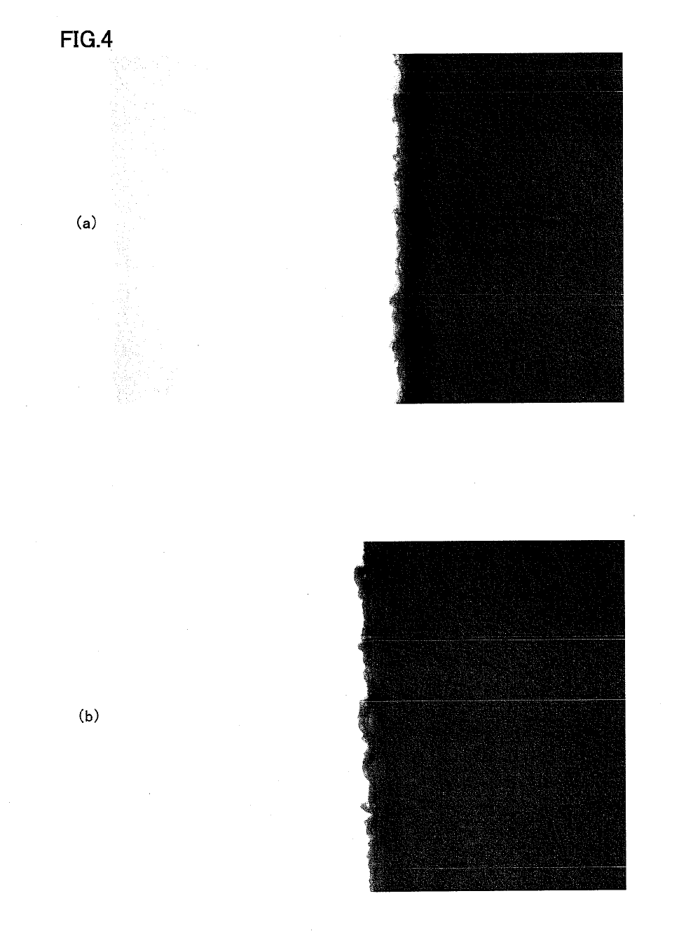

This vacuum pump was operated for several tens of minutes to completely remove the solvent within internal channel 22, thereby forming gas separation unit 20 provided with a stationary phase made of PEG 600 on the wall surface of internal channel 22 in the microcolumn. The cross section of gas separation unit 20 produced in this way was observed with the microscope to measure the thickness of the stationary phase, which showed that the stationary phase has a thickness of 1.0 μm. FIG. 4(a) shows an image of the cross section of the internal channel before being modified by the stationary phase that is taken by a microscope. FIG. 4(b) shows an image of the cross section of the internal channel after being modified by the stationary phase that is taken by the microscope.

### Example A2

The microcolumn according to the present example was produced by the method similar to that used in Example A1 except that polyethylene glycol having an average molecular weight of 200 and a relative permittivity of 18.43 (PEG 200: manufactured by GL Sciences, Inc.) was used as a material forming a stationary phase, as compared with the microcolumn in Example A1.

### Example A3

The microcolumn in the present example was produced by the method similar to that used in Example A1 except using a microcolumn of 6 cm square in which a groove as an internal channel having a width of 200 μm and a depth of 200 μm was formed in a meandering line and neighboring lines of the channel were arranged at a distance of 200 μm from each other (the internal channel having a length of about 10 m), as compared with the microcolumn in Example A1 .

### Example A4

The microcolumn in the present example was produced by the method similar to that used in Example A1 except using a microcolumn of 8 cm square in which a groove as an internal channel having a width of 200 μm and a depth of 200 μM was formed in a meandering line and neighboring lines of the channel were arranged at a distance of 200 μm from each other (the internal channel having a length of about 17 m), as compared with the microcolumn in Example A1 .

### Example A5

The microcolumn in the present example was produced by the method similar to that used in Example A1 except using a microcolumn of 6 cm square in which a groove as an internal channel having a width of 200 μm and a depth of 200 μm was formed in a meandering line and neighboring lines of the channel were arranged at a distance of 200 μm from each other (the internal channel having a length of about 10 m), as compared with the microcolumn in Example A1, and except using polyethylene glycol having an average molecular weight of 1000 and a relative permittivity of 9.05 (PEG 1000: manufactured by GL Sciences, Inc.) as a material forming a stationary phase.

### Comparative Example A1

As compared with the microcolumn in Example A1, the microcolumn in Comparative Example A1 was produced by the method similar to that used in Example A1 except using a stationary phase made of polyethylene glycol having an average molecular weight of 20000 and a relative permittivity of 7.7 (PEG 20M: manufactured by GL Sciences, Inc.). It is to be noted that PEG 20M is generally used for a commercially available capillary column having strong polarity.

<Study of Length of Internal Channel and Material of Stationary Phase>

As in Examples A1 to A5 and Comparative Example A1, studies have been conducted employing a gas chromatograph mass spectrometer (product name: JMS-K9 (manufactured by JEOL Ltd.)) to examine how the separation performance varied when the length of the internal channel and the material of the stationary phase in the microcolumn were changed. Specifically, a mixed liquid of acetone/ethanol/water at a mixing ratio of 1:1:100 was introduced into the GCMS to detect each gas component by the GCMS.

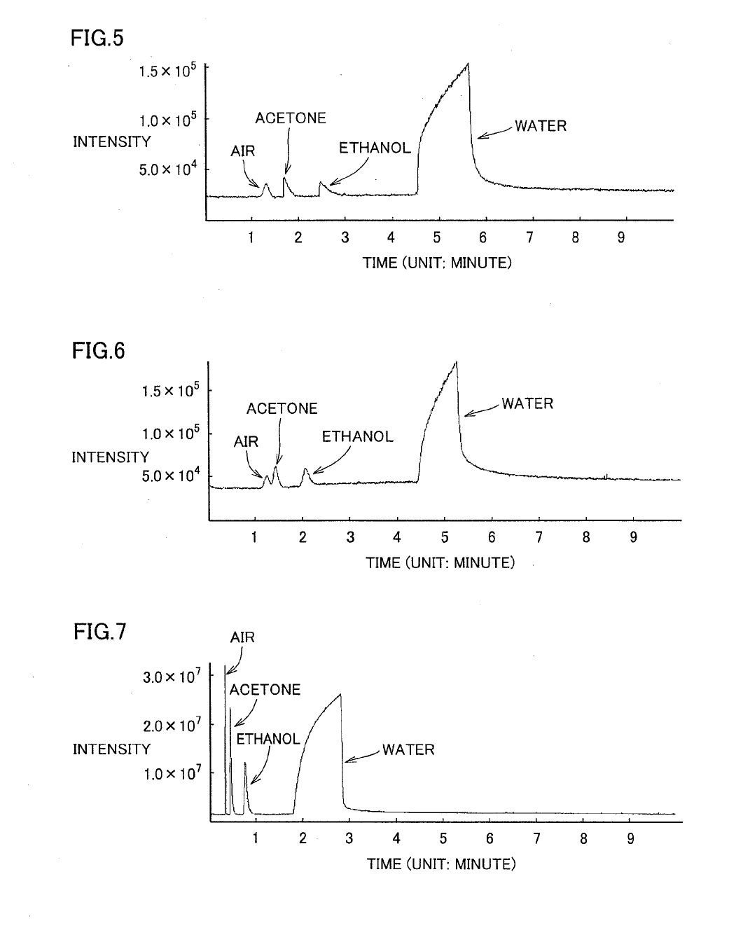

FIG. 5 is a chromatogram detected using the GCMS when a mixture gas of acetone/ethanol/water is introduced into the microcolumn produced in Example A1. The vertical axis in FIG. 5 represents the peak intensity of the detected component while the horizontal axis in FIG. 5 represents the retention time (minute) required to detect the component.

As apparent from the chromatogram in FIG. 5, the microcolumn in Example A1 shows each retention time as follows: air for 1 minute and 20 seconds, acetone for 1 minute and 45 seconds, ethanol for 2 minutes and 30 seconds, and water for 4 minutes and 30 seconds to 5 minutes and 50 seconds. This shows that the microcolumn in Example A1 can separate the components of the mixture gas of acetone/ethanol/water.

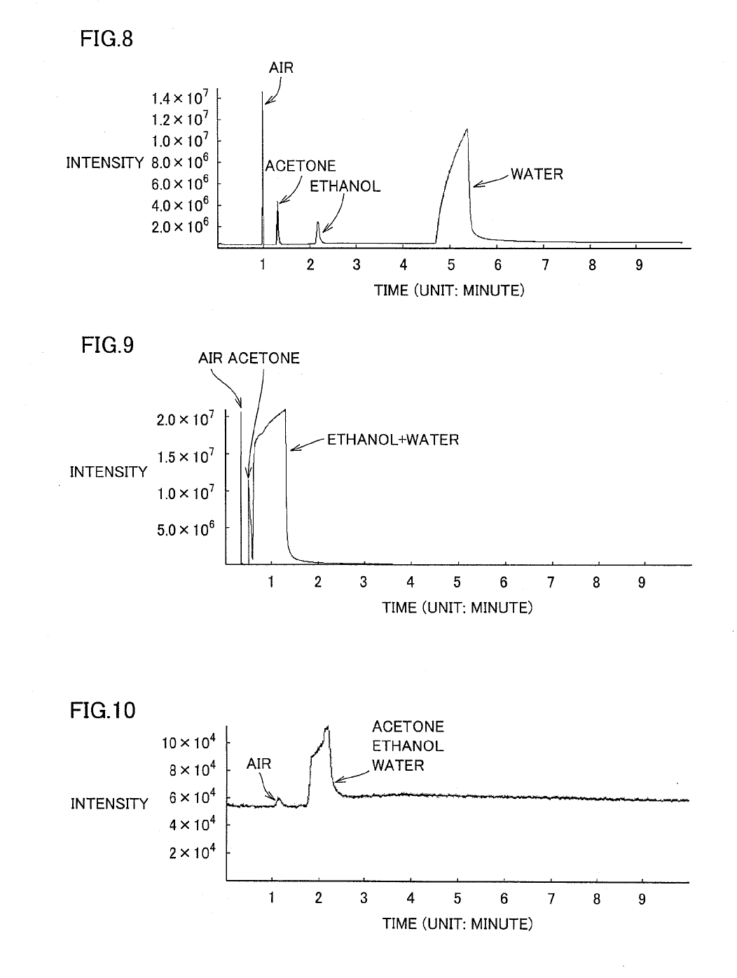

Similarly, FIGS. 6 to 9 each show a chromatogram detected using a GCMS when the mixture gas of acetone/ethanol/water is introduced into the microcolumn produced in Examples A2 to A5, respectively. Based on the results shown in FIGS. 6 to 9, it was found that the microcolumn in each of Examples A2 to A5 could separate at least the acetone component among the mixture gas of acetone/ethanol/water.

FIG. 10 is a chromatogram obtained when a mixture gas of acetone/ethanol/water is introduced into the microcolumn in Comparative Example A1. The chromatogram in FIG. 10 shows that the retention time of acetone, ethanol and water are overlapped with one another, which prevents separation of the components of the mixture gas of acetone/ethanol/water.

In this way, the microcolumn in each of Examples A1 to A5 could separate the components of the mixture gas whereas the microcolumn in Comparative Example A1 could not separate the components of the mixture gas. It is considered that this probably results from the length of the microcolumn and the composition constituting the stationary phase thereof. In other words, it is estimated that the entire length of the internal channel in the microcolumn was about 10 m, which was shorter than the length of the conventionally used capillary column of about 30 m, with the result that PEG 20M having a low relative permittivity of less than 10 could not sufficiently function as a stationary phase, so that sufficient separation could not be achieved.

### Example 1

In the present example, the gas analyzer shown in FIG. 1 was manufactured by the following procedure. The microcolumn in Example A1 described above was used as gas separation unit 20 while a manual gas sampler for gas chromatograph (manufactured by GL Sciences, Inc.) was used as gas introduction unit 10. The manual gas sampler for gas chromatograph referred herein (which will be hereinafter also referred to as a “gas sampler”) includes first channel 12 for introducing a sample gas, second channel 13 for discharging a part of the introduced sample gas through the gas discharge port, third channel 15 for introducing a carrier gas, fourth channel 16 for supplying the sample gas into gas separation unit 20, and gas storage unit 19 for storing the sample gas.

Fourth channel 16 of the gas sampler and gas separation unit 20 were connected to each other using a reducing union of 1/16×0.25. This allowed the carrier gas introduced through third channel 15 of the gas sampler to be introduced into microcolumn 21 in gas separation unit 20 through fourth channel 16.

Then, one end of the capillary tube was inserted into microcolumn 21 in gas separation unit 20, for connection therebetween. The other end of the capillary tube was connected to the vicinity of gas sensor 31 in gas detection unit 30, that is, connected such that the other end of the capillary tube was located at a distance of 1.5 mm from gas sensor 31, to produce gas detection unit 30. The gas analyzer was manufactured in this way.

### Examples 2 to 5

In Examples 2 to 5, the gas analyzer in each of Examples 2 to 5 was manufactured by the method similar to that used in Example 1 except that the microcolumn in Example A1 was replaced with the microcolumn in each of Examples A2 to A5 described above.

### Comparative Example 1

In Comparative Example 1, the gas analyzer was manufactured by the method similar to that used in Example 1 except that the microcolumn in Example A1 was replaced with the microcolumn in Comparative Example A1.

<Detection of Sample Gas>

The semiconductor sensor of the gas analyzer manufactured in Example 4 was used to examine whether or not acetone could be detected. Specifically, acetone was diluted with air to set its concentration at 100 ppb, 250 ppb, 500 ppb, 1000 ppb, and 4800 ppb to obtain a sample gas that was then introduced from the gas introduction unit.

(1) Sample gas: acetone

(2) Introducing amount of sample gas: 50 μL

(3) Carrier gas: air, introduction pressure of 0.26 MPa

(4) Temperature of microcolumn: room temperature (25° C.)

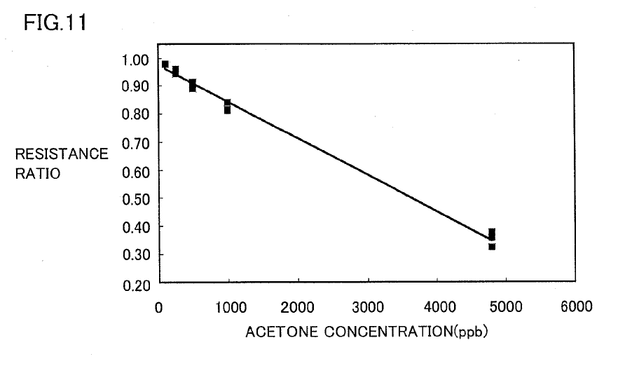

FIG. 11 is a graph graphically showing an output of the resistance change detected by a gas sensor. As shown in FIG. 11, as the acetone concentration is increased, its resistance ratio is decreased in a linear function manner. This apparently shows that the gas analyzer in the present example can accurately detect acetone.

<Relationship between Flow Velocity of Sample Gas and Component Separation Performance>

The gas analyzer manufactured in Example 3 was used to check the performance of component separation at the time when the flow rate of the sample gas introduced into the microcolumn was changed. Specifically, a needle valve was prepared in order to adjust the flow rate at which the sample gas was introduced into the microcolumn. Then, the pressure at which the sample gas was introduced into a microcolumn was adjusted by the needle valve in three stages such as 0.04 MPa, 0.11 MPa and 0.26 MPa, to check the component separation performance of the microcolumn at each pressure. In addition, the sample gas used herein contained 1 ppm of ethanol and 1 ppm acetone, in which case clean air was used as a base gas.

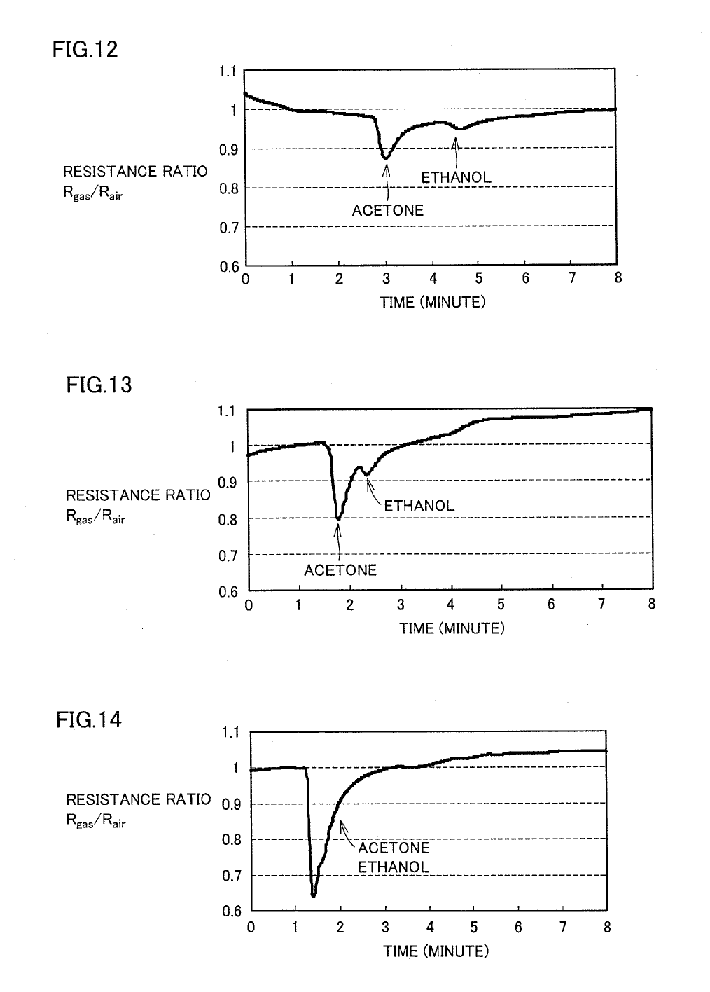

FIGS. 12 to 14 each represents a graph showing the output of the resistance change of the gas sensor at the time when the sample gas was introduced at pressures of 0.04 MPa, 0.11 MPa and 0.26 MPa, respectively. As apparent from the graph in each of FIGS. 12 to 14, the components of acetone and ethanol could be separated when the sample gas was introduced at a pressure of 0.04 MPa or 0.11 MPa, whereas the components of acetone and ethanol could not be separated when the sample gas was introduced at a pressure of 0.26 MPa. This apparently shows that, in order to separate components using a microcolumn, consideration should be given also to the factors of pressure applied when introducing the sample gas into the microcolumn.

<Detection of Mixture Gas>

It was examined how the gas analyzer in Example 4 conducted detection when a sample gas containing ethanol and acetone mixed together was introduced. Specifically, nitrogen gas containing 1 ppm of acetone and clean air containing 1 ppm of ethanol were separately introduced into the gas analyzer, to measure the retention time required to detect each component. Then, the clean air containing 1 ppm of ethanol and 1 ppm of acetone was introduced into the gas analyzer, to check how each component was detected.

In addition, in the case of any of the above-described sample gases, the first state was switched to the second state after one minute since introduction of the sample gas, and the second state was kept for two seconds, and then, switched to the first state, for introducing the sample gas into the microcolumn. The pressure applied when introducing the sample gas in this case was set at 0.26 MPa.

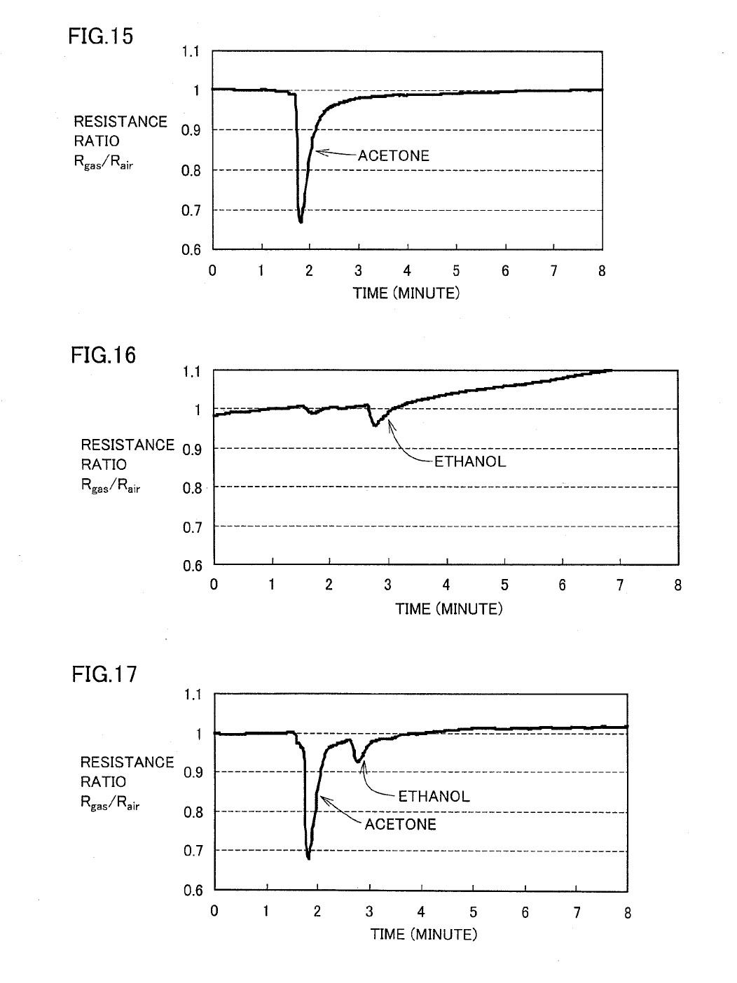

FIG. 15 represents a graph showing the output of the resistance change at the time when a nitrogen gas containing 1 ppm of acetone was introduced. The graph in FIG. 15 shows the peak of the resistance change at 1 minute and 48 seconds. Accordingly, it was found that the retention time of acetone was 48 seconds which was obtained by subtracting the time period during which the channel switching mechanism was operated after 1 minute since introduction of the sample gas.

On the other hand, FIG. 16 represents a graph showing the output of the resistance change at the time when the clean gas containing 1 ppm of ethanol was introduced. The graph in FIG. 16 shows the peak of the resistance change at 2 minutes and 45 seconds. Accordingly, it was found that the retention time of ethanol was 1 minute and 45 seconds.

FIG. 17 is a graph showing an output of the resistance change at the time when the sample gas containing 1 ppm of ethanol and 1 ppm of acetone is introduced into the gas analyzer in Example 4. The graph in FIG. 17 shows the peak of the resistance change each at 1 minute 49 seconds and 2 minutes 46 seconds. Thus, the results in FIGS. 15 and 16 described above show that the peak at 1 minute and 49 seconds is a peak of acetone while the peak at 2 minutes and 46 seconds is a peak of ethanol.

The result in FIG. 17 apparently shows that, when the sample gas containing ethanol and acetone was introduced into the gas analyzer, the components of ethanol and acetone could be separated and detected for each component.

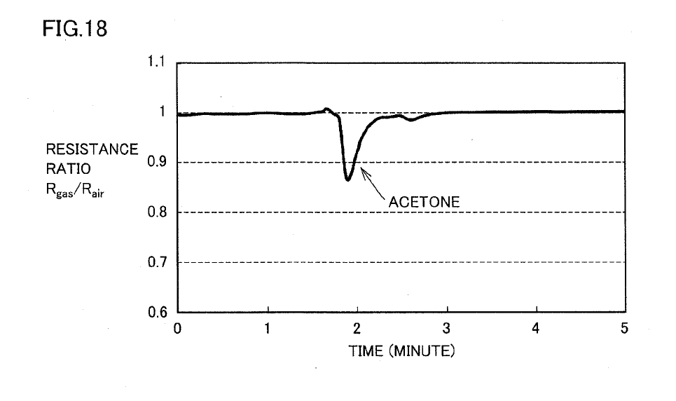

Exhaled breath was introduced into the gas analyzer manufactured in Example 4, thereby detecting the concentration of acetone contained in the exhaled breath. First, the pressure of the carrier gas (air) introduced into the microcolumn was adjusted by a needle valve at 0.26 MPa. Then, the first state was switched to the second state after 1 minute since starting circulation of the exhaled breath, and this second state was kept for two seconds, and then, switched to the first state, thereby introducing 50 μl of exhaled breath of room temperature into the microcolumn. FIG. 18 is a graph showing an output of the resistance change at the time when exhaled breath is introduced as a sample gas into the gas analyzer in Example 4. The graph in FIG. 18 shows the peak of the resistance change at 1 minute and 48 seconds, in which case the resistance ratio is 0.86. Based on this, it was found that 50 μl of exhaled breath contains 0.8 ppm of acetone.

<Change over Time of Separation Performance of Microcolumn>

It was examined how the separation performance of the microcolumn varied when the microcolumns in Example A1 and Example A2 each were used in the GCMS continuously for 30 days.

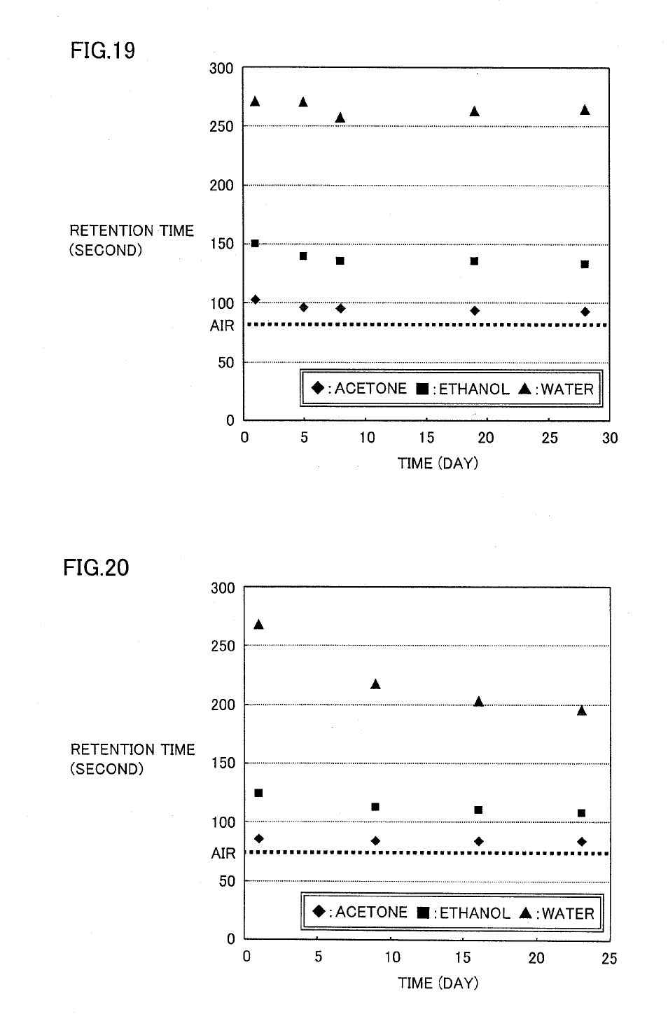

FIG. 19 is a graph showing a change over time of component separation at the time when a mixture gas is separated by the GCMS using the microcolumn in Example A1. The vertical axis in FIG. 19 shows the retention time (second) of each component while the horizontal axis in FIG. 19 shows the number of days (day) since the start of continuous use of the analyzer. FIG. 19 is the plot of the peak of the retention time of each component in the case of introduction on the first day, the fifth day, the eighth day, the 19th day and the 29th day since the start of introduction of the mixture gas into the microcolumn in Example A1 .

The result in FIG. 19 shows that, despite the continuous operation for 30 days, the microcolumn in Example A1 (a stationary phase made of PEG 600) does not cause a significant change in the position of its peak. Based on this, it is also found that the microcolumn in Example A1 has a component separation performance that is less likely to deteriorate.

FIG. 20 is a graph showing a change over time of component separation at the time when a mixed gas is separated using the microcolumn in Example A2. FIG. 20 is the plot of the peak of the retention time of each component at the time when the mixture gas is introduced on the ninth day, the 16th day and the 23rd day since the start of continuous operation of the microcolumn in Example A2.

The result in FIG. 20 shows that the retention time for water is reduced with time in the case of the microcolumn in Example A2 (the stationary phase made of PEG 200). This apparently shows that the microcolumn in Example A2 has a component separation performance that tends to deteriorate with time.

In this way, the microcolumn in Example A1 has a separation performance that is less likely to deteriorate as compared with the microcolumn in Example A2. This is considered because there is a tendency that the less the molecular weight of PEG is, the higher the polarity is and the lower the viscosity is. In other words, it is considered that PEG 200 tends to have a relatively low viscosity due to its lower molecular weight, and therefore, hard to be retained on the wall surface of internal channel 22, with the result that a part of PEG 200 flows out during the continuous operation.

It is also considered that the separation performance of the microcolumn using PEG 600 was not deteriorated because the retaining amount onto the inner wall of the channel was not changed due to PEG 600 having a relatively low polarity but having a relatively high viscosity.

In the present invention, an explanation has been given in the above with regard to the suitable embodiments of the gas analyzer, which is not limited to the above description, but can be configured in the manner other than those described above.

Although the embodiments and examples according to the present invention have been described as above, the configurations of the embodiments and examples described above are intended to be combined as appropriate from the beginning.

It should be understood that the embodiments and examples disclosed herein are illustrative and non-restrictive in every respect. The scope of the present invention is defined by the terms of the claims, rather than the description above, and is intended to include any modifications within the scope and meaning equivalent to the terms of the claims.

### INDUSTRIAL APPLICABILITY

According to the present invention, a gas component detecting device can be provided that is compact, effective in facilitating preventive healthcare and suitable for personal use.

### REFERENCE SIGNS LIST

10 gas introduction unit, 11 gas introduction port, 12 first channel, 13 second channel, 15 third channel, 16 fourth channel, 17 pressure adjusting means, 18 channel switching mechanism, 19 gas storage unit, 20 gas separation unit, 21 microcolumn, 22 internal channel, 25 airflow generating means, 30 gas detection unit, 31 gas sensor, 40 gas sampling unit, 41 check valve.

1. A gas analyzer comprising:
a gas introduction unit including a gas introduction port for introducing a sample gas;
a gas separation unit including a microcolumn for separating components of the sample gas supplied from said gas introduction unit; and
a gas detection unit detecting a gas component separated by said gas separation unit,
said microcolumn being provided with an internal channel having a wall surface modified by a stationary phase, and
said stationary phase being made of a polar material having a relative permittivity of not less than 10 at 30° C.

2. The gas analyzer according to claim 1, wherein said polar material is made of polyethylene glycol having an average molecular weight of not less than 200 and not more than 1000.

3. The gas analyzer according to claim 1, wherein an inner diameter of said internal channel is defined as D and a thickness of said stationary phase is defined as t, which leads to a condition that 0.005≦t/D≦0.02.

4. The gas analyzer according to claim 1, wherein said stationary phase has a thickness of not less than 1 μm and not more than 2 μm.

5. The gas analyzer according to claim 1, wherein said sample gas contains acetone.

6. The gas analyzer according to claim 1, wherein
said gas detection unit is provided therein with a gas sensor for detecting a detection gas, and
said gas sensor is disposed near an outlet port for the gas component separated by said gas separation unit.
